(19) United States
(12) Patent Application Publication
(10) Pub. No.: US 2022/0044763 A1
(43) Pub. Date: Feb. 10, 2022

(54) MOLECULAR ENCODING AND COMPUTING METHODS AND SYSTEMS THEREFOR

(71) Applicant: Tahereh Karimi, Houston, TX (US)

(72) Inventor: Tahereh Karimi, Houston, TX (US)

(21) Appl. No.: 17/275,853

(22) PCT Filed: Sep. 13, 2019
(86) PCT No.: PCT/US19/51160

§ 371 Date: Mar. 12, 2021

Related U.S. Application Data
(60) Provisional application No. 62/731,859, filed on Sep. 15, 2018.

(51) Int. Cl. G16B 30/00; G16B 15/10

(52) U.S. Cl. CPC G16B 30/00; G16B 15/10

(57) ABSTRACT

The present disclosure relates to methods of data encryption and data storage using molecular systems. The present disclosure also relates to molecular systems and methods for solving a polynomial time problem. Benefits of the methods and systems disclosed herein can include providing for the secure storage and retrieval of large amounts of encrypted data in a stable molecular system having random-access capability. Benefits of the methods and systems disclosed herein can include providing molecular computing systems that can solve complex polynomial time problems.

### REFERENCE TO RELATED APPLICATIONS

This application claims the benefit of the priority date of U.S. Provisional Patent Application No. 62/731,859, filed Sep. 15, 2018, which is incorporated by reference in its entirety.

### TECHNICAL FIELD

The present disclosure relates to methods of data encryption and data storage using molecular systems. The present disclosure also relates to molecular systems and methods for solving a polynomial time problem. Benefits of the methods and systems disclosed herein can include providing for the secure storage and retrieval of large amounts of encrypted data in a stable molecular system having random-access capability. Benefits of the methods and systems disclosed herein can include providing molecular computing systems that can solve complex polynomial time problems.

### BACKGROUND

Information technology has seen explosive growth in recent years. A vast amount of data is transferred electronically on a daily basis, whether through email, e-commerce, online banking, or any of a myriad of purposes. Some of this information is of a sensitive or confidential nature. As the digital world continues to grow exponentially, the need for secure ways to protect the confidentiality of sensitive information grows as well. Cybernetic attacks attempting to intercept, and capture information transferred over the internet pose a constant threat to the security and integrity of data transmission worldwide. Data encryption is one method used to secure transmitted information. Techniques of encoding data before it is sent through various communication channels have been and continue to be developed; but with the number of threats to data security continuing to increase, there remains a need for improved ways of making data communications unreadable to all but the intended recipients.

Conventional computer methods store data in a binary format in the form of series of 0 and 1 digits. Cryptography methods help increase the security of data communications by encoding data in a binary format using an encryption key, to make the data unreadable without the use of the correct decryption key. If an attacker discovers the key, the data becomes readable.

Encryption keys of increasing sophistication have been developed to combat the threat to data security. One area in which such developments have been made is in the field of molecular computing. Data encoding techniques making use of the biological genetic coding system of deoxyribonucleic acid (DNA) have provided encrypted data storage and retrieval systems with feasibility for encoding and storing data with increased levels of coding complexity. However, the need remains for the means to store ever increasing amounts of data securely, and the ability to safely access desired pieces of information selectively from among massive amounts of stored information.

The field of operations research, also referred to as management science, seeks to apply advanced analytical methods to improve problem solving and decision making relevant to management, economics, business, engineering, and management consulting, among other fields. Optimal solutions to complex decision problems are sought by the use of mathematical modeling and complex computations. An example of such a complex problem is the travelling salesman problem (TSP), which asks the question: given a list of cities and the distances between each pair of cities, what is the shortest possible route that visits each city and returns to the origin city? This problem may sound academic but airlines and package delivery services struggle with this issue every day. Solutions to TSP problems are among the most elusive in computer science history. There remains a need for methods for solving such complex computational problems for the benefit of operational decision making.

### SUMMARY

Embodiments herein are directed to methods of data encryption and data storage using molecular systems. In an embodiment, a method of recording and reading a binary code is disclosed. In various embodiments, the method includes providing a binary code; creating a recording key by assigning at least two amino acids a binary code identity; recording the binary code into at least one coded polypeptide by adding the at least two amino acids in sequence to form a coded peptide sequence according to the recording key, wherein the coded peptide sequence corresponds to the binary code; determining the coded peptide sequence by mass spectroscopy; and reading the coded peptide sequence into the binary code by identifying the at least two amino acids according to their binary code identity. In an embodiment, from two to sixteen amino acids are assigned a binary code identity. In an embodiment, the at least one coded polypeptide sequence is formed by chemical-based peptide synthesis, by in vitro translation of at least one recombinant polynucleotide sequence encoding the at least one coded peptide sequence, or a combination thereof.

In an embodiment, the present method includes identifying at least one target peptide sequence in the at least one coded polypeptide, wherein the at least one target peptide sequence includes at least one detectable label on a polypeptide N-terminus, at least one detectable label on a polypeptide C-terminus, at least one nucleotide recognition sequence, at least one protease recognition sequence, or a combination thereof and determining the target peptide sequence by mass spectroscopy. In certain embodiments, the at least one nucleotide recognition sequence includes a TALE identification sequence, a zinc finger sequence, a CRISPR recognition sequence, or a combination thereof. In such embodiments, the method includes providing at least one labeled nucleotide recognizing the at least one nucleotide recognition sequence; and identifying a target peptide sequence in the coded peptide sequence by hybridizing the at least one labeled nucleotide to the at least one nucleotide recognition sequence.

In certain embodiments, the method includes storing the at least one coded polypeptide as a lyophilized powder, in a liquid buffer, immobilized on a microarray, or a combination thereof.

In an embodiment, the method provides including at least one nucleotide-binding sequence in the at least one coded polypeptide; immobilizing the at least one coded polypeptide on at least one position in a microarray; providing at least one detectably labeled polynucleotide recognized by the at least one nucleotide-binding sequence; and identifying at least one target coded polypeptide by hybridizing the at least one detectably labeled polynucleotide to the at least one nucleotide-binding sequence. In certain embodiments, the at least one nucleotide-binding sequence includes a TALE identification sequence, a zinc finger sequence, a CRISPR recognition sequence, or a combination thereof. In certain embodiments, the at least one detectably labeled polynucleotide includes a molecular label. In certain embodiments, immobilizing the at least one coded polynucleotide on at least one position in a microarray includes a streptavidin-biotin bond, a polyhistidine tag bound to a silicon, glass, or a metal chip surface, or a combination thereof.

Embodiments herein disclose a polypeptide storage system including at least one coded polypeptide made by embodiments of the methods herein.

An embodied method of recording and reading a binary code herein includes providing a binary code; creating a recording key by assigning at least two amino acids or at least two nucleic acid residues a binary code identity; recording the binary code into at least one coded polypeptide or at least one polynucleotide by adding the at least two amino acids or the at least two nucleic acid residues in sequence to form a coded peptide sequence or a coded nucleotide sequence according to the recording key, wherein the coded peptide sequence or the coded nucleotide sequence corresponds to the binary code; determining the coded peptide sequence or the coded nucleotide sequence by mass spectroscopy; and reading the coded peptide sequence or the coded nucleotide sequence into the binary code by identifying the at least two amino acids according to their binary code identity.

The present disclosure relates to systems and methods for solving a polynomial time problem using a molecular based system. In an embodiment, a system for solving a polynomial time problem includes a polynomial time problem and a map, wherein the map includes N number of map locations with a distance between their map locations; and a closed loop molecular structure having a number N of nodes located along the closed loop molecular structure, wherein each of the N nodes corresponds to a different map location, wherein each of the N nodes is connected to a different node by an oligomer containing chain, wherein each of the nodes is connected to N−1 different single stranded oligonucleotide identification sequences, wherein each single stranded oligonucleotide identification sequence contains an identification portion, wherein the identification portion contains a sequence which corresponds to an identity of the node to which it is attached, and an interaction portion, which is complementary to one single stranded oligonucleotide identification sequence on another node, wherein each pair of single stranded oligonucleotide identification sequences that is capable of hybridizing with its complementary single stranded oligonucleotide identification sequence, to form a double stranded oligonucleotide identification sequence between a pair of nodes, has a length corresponding to the distance between the map location of the pair of nodes.

In various embodiments of a system for solving a polynomial time problem, the single stranded oligonucleotide identification sequences include single stranded DNA, a RNA, a single stranded polymer, or combinations thereof. In certain embodiments, the oligomer includes amino acids, nucleic acids, polyethylene glycol, an acrylate polymer, a water-soluble polymer, or combinations thereof. In certain embodiments, the closed loop molecular structure is dissolved in an aqueous buffer solution containing at least one polar buffer, a hydrogel, or a combination thereof. In certain embodiments, the nodes include polymer microbeads, carbon nanotubes, carbon nanoparticles, polypeptides, and combinations thereof. In certain embodiments, the nodes are connected to the single stranded oligonucleotide identification sequences including a streptavidin-avidin bond, an overlapping polynucleotide handle, or a combination thereof. In certain embodiments, at least one oligomer containing chain includes at least one restriction enzyme recognition site, at least one protease cleavage site, or combination thereof.

Embodiments herein provide methods of solving a polynomial time problem. In an embodiment, a method includes providing a polynomial time problem, a map, and a closed loop molecular structure of a system for solving a polynomial time problem disclosed herein, wherein the molecular structure is in an aqueous buffer solution; forming double stranded oligonucleotide identification sequences between the nodes; heating the aqueous buffer solution at a heating rate to a measurement temperature; adding a double stranded detection molecule to the aqueous buffer at a measurement time; sequencing the double stranded oligonucleotide identification sequences present at the measurement time by mass spectroscopy to provide the sequences of the identification portions of a pair of nodes; correlating the sequence of the identification portion to the pair of nodes they identified; quantifying an amount of the identification portions for each pair of nodes; and generating an answer to the polynomial time problem by correlating the amount of the identification portions for each pair of nodes present at the measurement time with the answer to the polynomial time problem.

In some embodiments, the method includes providing at least two sample vessels containing the molecular structure in an aqueous buffer solution; forming double stranded oligonucleotide identification sequences between the nodes in the at least two sample vessels at room temperature, wherein the double stranded oligonucleotide identification sequences include at least one nucleotide-binding sequence selected from a TALE identification sequence, a zinc finger sequence, a CRISPR recognition sequence, or a combination thereof, wherein the double stranded detection molecule is selected from a TALE DNA recognition domain, a zinc finger, a CRISPR-cas9 recognition domain, or a combination thereof; and sequencing the double stranded oligonucleotide identification sequences present in the at least two sample vessels at the least two measurement times by mass spectroscopy to provide the sequences of the identification portions of pairs of nodes.

In an embodiment, the method includes labeling the double stranded detection molecule before adding the double stranded molecule to the aqueous buffer at the measurement time; and detecting a signal from the labeled double stranded detection molecule before sequencing the double stranded oligonucleotide identification sequences present at the measurement time.

### BRIEF DESCRIPTION OF THE DRAWINGS

The foregoing summary, as well as the following detailed description of the embodiments, will be better understood when read in conjunction with the attached drawings. For the purpose of illustration, there are shown in the drawings some embodiments, which may be preferable. It should be understood that the embodiments depicted are not limited to the precise details shown. Unless otherwise noted, the drawings are not to scale.

FIG. 1 is a flow chart depicting an embodiment of recording and reading a binary code disclosed herein.

FIG. 2 is an illustration of an embodiment of methods of recording and reading a binary code disclosed herein.

FIG. 3 is an illustration of an embodiment of methods of recording and reading a binary code disclosed herein.

FIG. 4A is a schematic diagram depicting an example polynomial time problem (Traveling Salesman Problem).

FIG. 4B is an illustration of an embodiment of a closed loop molecular structure for solving a polynomial time problem disclosed herein.

### DETAILED DESCRIPTION

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object. For example, “at least one coded polypeptide” means one coded polypeptide, more than one coded polypeptide, or any combination thereof.

Unless otherwise noted, the term “about” refers to ±10% of the non-percentage number that is described, rounded to the nearest whole integer. For example, about 100 mm, would include 90 to 110 mm. Unless otherwise noted, the term “about” refers to ±5% of a percentage number. For example, about 20% would include 15 to 25%. When the term “about” is discussed in terms of a range, then the term refers to the appropriate amount less than the lower limit and more than the upper limit. For example, from about 100 to about 200 mm would include from 90 to 220 mm.

Unless otherwise noted, properties (height, width, length, ratio etc.) as described herein are understood to be averaged measurements.

Unlike human-made computers that are operated according to physical and electrical based coding, biological systems use unique chemical-based coding systems to encode information. Biological information is embedded in storage materials such as genetic information in DNA, or is encoded through ordered chemical interactions between molecules, such as during protein translation. DNA, the genetic material that carries all of the information needed for the formation of any individual organism from one generation to the next, is the most well-known biologically coded material utilized by nature. DNA can provide a huge storage capacity compared to computer systems, in part because DNA encodes data using four distinct subunits (Adenine: A, Guanine: G, Cytosine: C and Thymine: T), while current man-made computers use only a binary (0, 1) coding system. Other classes of sequence-based biological coding systems include messenger Ribonucleic Acid (mRNA), another 4 unit, temporary coding sequence which is used in the cell to translate DNA codes to direct protein synthesis in the cell, and peptide/protein sequences, which are composed of 20 commonly used amino acid units and dictate the structure and function of cellular proteins.

Biological systems often apply multiple layers of “primary” cellular coding systems which in turn lead to more complex “coding” that can take place throughout the body. In primary cellular coding, each layer of coding involves different types of coding subunits, such as the multilayer coding to produce proteins within the cell using the coding languages of DNA, mRNA, amino acids and peptides/proteins. More advanced coding takes place through protein-protein interactions, intracellular signaling pathways, systemic signaling pathways (such as through the endocrine system), and more restricted signaling pathways, such as the neural network interactions directed by neurotransmitters.

The use of biological coding systems in the development of polymer-based coding has become an emerging subject in both data storage and material science. Initially, DNA was applied as a coding medium for non-biological data. Deoxyribonucleic acid (DNA)-based data storage systems have been developed and serve to demonstrate the feasibility of biological data storage. Different types of synthetic polymers have also been used for data storage. Many problems, however, remain to be solved with DNA-based and synthetic polymer-based data storage systems. One technical challenge is in addressing the need for storage of ever greater quantities of encrypted data, and more complex methods of encryption to protect data integrity and confidentiality. Most of the current polymer-based coding systems are composed of two coding sub-units which convert electronic-based data into chemical-based coding. Although DNA presents an increased coding capacity with its 4 subunits compared to the binary system, there remains a need for systems with greater coding capacity and encryption complexity.

Another challenge is the limitations in chemical and structural stability of DNA-based coding systems. DNA has susceptibilities to environmental, chemical or enzymatic degradation, resulting in a requirement for cold storage of DNA based data. Mutations in the structure of nucleic acids can also occur during DNA replication processes.

Another primary obstacle includes the inability to randomly access pieces of information encoded in DNA or synthetic polymers; recovering stored data on a large scale currently requires the sequencing of full data, even if only a subset of the information needs to be extracted. Recent developments include a primer-based method for random access to DNA based information. This method is based on providing copies of information from the original information which is stored in DNA applying Polymers Chain Reaction (PCR). This method can be applied only for DNA based information storage, however. A major challenge to the broad application of the current natural or synthetic polymer-based coding systems remains the limitations in random data access. For example, MICROSOFT® biological computing systems offer computing data storage, but such systems do not currently offer random data access.

Embodiments of the present disclosure can provide methods and systems for recording and reading a binary code. Various embodiments herein can provide methods of recording a binary code into at least one coded polypeptide, by adding at least two amino acids in sequence to form a coded peptide sequence according to a recording key, wherein the codes peptide sequence corresponds to the binary code. Such embodiments can provide a benefit of data encoded in a polymer sequence having greater structural and chemical stability and resistance to potential errors in coding due to mutations. Such embodiments can also provide a benefit of greater data storage capabilities, including up to Exabyte data storage capabilities with dramatically lower energy and space requirements. Such embodiments can provide data storage capabilities with greater data integrity. Such embodiments can provide a benefit of greater encryption capability, thus increasing the security of encoded data. Such embodiments can provide a benefit of multiple layers of data encryption, thus providing increased security of encoded data.

Various embodiments of the methods and systems herein can also provide a benefit of random access to encoded data, via providing a multi-layer molecular coding system. For example, the disclosed methods can identify at least one target peptide sequence in at least one coded polypeptide, by hybridizing a labeled nucleotide to at least one target peptide sequence having at least one nucleotide recognition sequence that is specifically recognized by the labeled nucleotide.

Increasingly advanced analytical and mathematical methods are needed to provide solutions to complex problems and the making of decisions relevant to management, economics, business, engineering, and management consulting, among other fields. An example of such a complex problem is the travelling salesman problem (TSP), which asks the question: given a list of cities and the distances between each pair of cities, what is the shortest possible route that visits each city and returns to the origin city? One approach to solving this type of problem is to generate all possible routes, and then determine which possible route is the shortest, and therefore likely the least costly. Current computer operations may be used to process the possible routes sequentially where processing time is acceptable; however, solutions to TSP problems can be so complex that a supercomputer would take years to solve them. Analysis of cost-effective travel routes is of great commercial importance. For example, solving the TSP problem could find direct application for the complex air travel routes for commercial airlines or the drone delivery of goods. The mathematical community has long recognized the TSP problem as a major mathematical challenge for the modern era (including Artificial Intelligence). There remains a need for methods for solving such complex computational problems. Embodiments disclosed herein can provide a benefit of systems and methods for solving a polynomial time problem. For example, an embodiment of a system herein can provide a closed loop molecular structure that can simulate and help to determine an optimal solution to a polynomial time problem, such as a TSP problem. Embodiments herein can provide a benefit of automated systems and methods for solving a polynomial time problem.

One benefit of the presently disclosed method is that the method can be performed at temperatures from about 15° C. to about 90° C. In contrast, many quantum computing applications required low temperatures near −273° C., which is costly in terms of equipment and power. Even traditional computers require near room temperature heating and cooling costs. The presently disclosed methods are able to function at ambient temperatures in all but the most extreme environments, so long as the enzymes are still capable of functioning.

### Embodiments of Methods of Recording and Reading a Binary Code

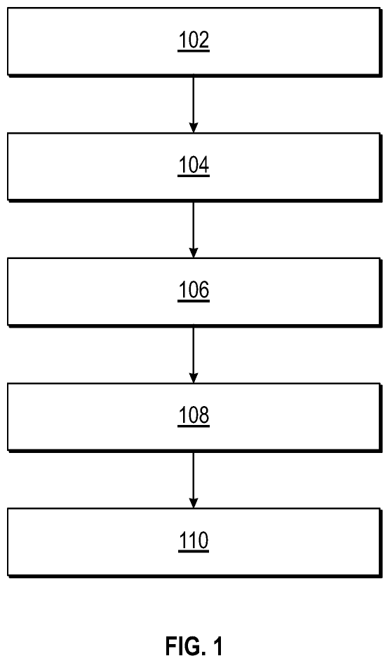

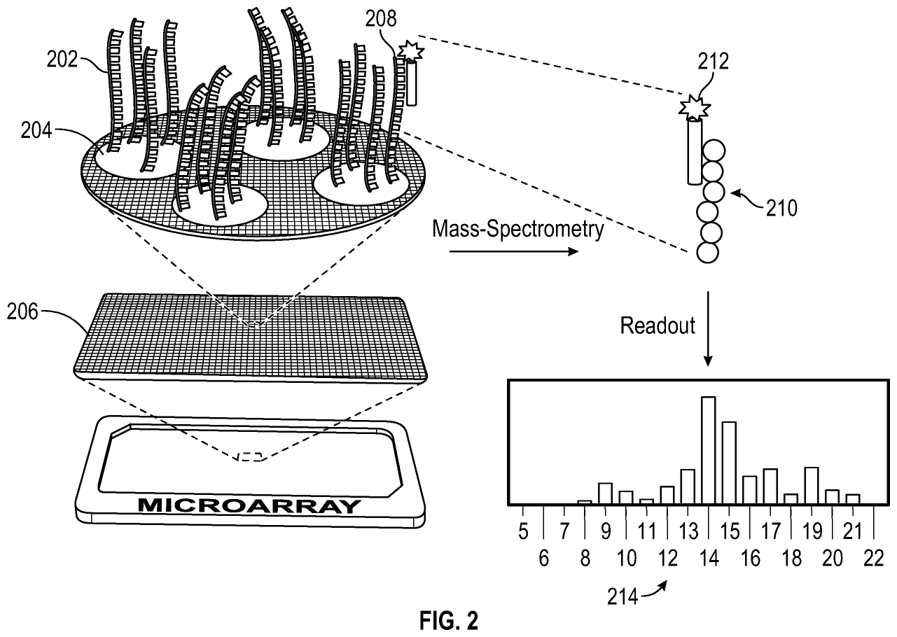

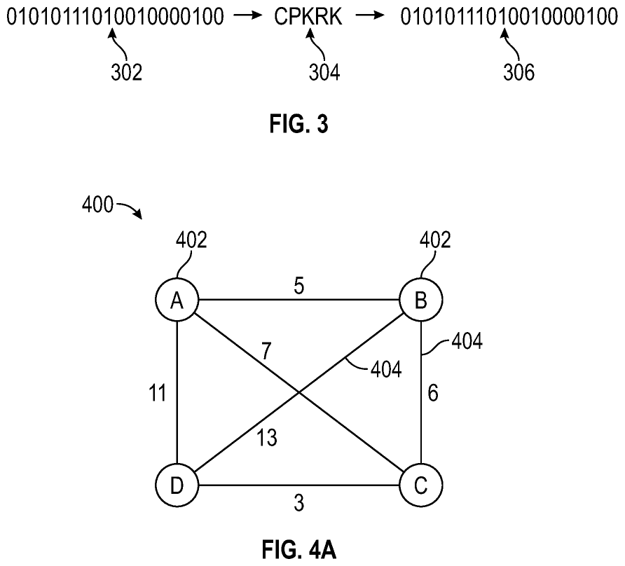

The present disclosure relates to a method of recording and reading a binary code, including converting the binary coding to a molecular coding system that simulates or is analogous to the amino acid coding system. As a general overview of a method disclosed herein, referring to FIG. 1, the method includes 102 providing a binary code; 104 creating a recording key by assigning at least two amino acids a binary code identity; 106 recording the binary code into at least one coded polypeptide by adding the at least two amino acids in sequence to form a coded peptide sequence according to the recording key, wherein the coded peptide sequence corresponds to the binary code; 108 determining the coded peptide sequence by mass spectroscopy; and 110 reading the coded peptide sequence into the binary code by identifying the at least two amino acids according to their binary code identity. As an illustration of a method disclosed herein, referring to FIG. 2, coded polypeptides 202 can be immobilized on positions 204 of microarray 206. Target peptide sequence 208 includes nucleotide recognition sequence 210 that is recognized by and hybridized to labeled nucleotide 212 to identify the target peptide sequence. The target peptide sequence is decoded by mass spectroscopy to allow readout 214 of the coded peptide sequence into the binary code, by identifying the amino acids in the coded peptide sequence according to their binary code identity. As an illustration of a method disclosed herein, referring to FIG. 3, binary code 302 is recorded into coded peptide sequence 304 according to the recording key shown in Table 1 of Example 1 below, and read into binary code 306 by identifying the amino acids according to their binary code identity.

Embodiments herein are directed to methods of data encryption and data storage using molecular systems. In an embodiment, a method of recording and reading a binary code is disclosed. In various embodiments, the method includes providing a binary code; creating a recording key by assigning at least two amino acids a binary code identity; recording the binary code into at least one coded polypeptide by adding the at least two amino acids in sequence to form a coded peptide sequence according to the recording key, wherein the coded peptide sequence corresponds to the binary code; determining the coded peptide sequence by mass spectroscopy; and reading the coded peptide sequence into the binary code by identifying the at least two amino acids according to their binary code identity. In an embodiment, from two to sixteen amino acids are assigned a binary code identity. Because of the wide variety in the structure of amino acids, differentiation between amino acids can be done with high resolution. By increasing the number of coding amino acid subunits with their structural diversity, embodiments herein can decrease the rate of coding errors related to environmental conditions, such as mutations. Such embodiments can provide benefits of increased biological based data storage capacity and more effective data encryption than is available with DNA based systems.

In an embodiment, the at least two amino acids include at least one β-type amino acid. Such an embodiment can provide a benefit of a coded peptide sequence having a highly stable chemical structure, and that can be resistant to bacterial proteases. In some embodiments, a molecular coding system as disclosed herein can include one or more natural amino acids or nucleic acids, including but not limited to one or more α-type amino acids. In some embodiments, a molecular coding system as disclosed herein can include one or more synthetic amino acids, or one or more polymers that mimic the properties of behavior of DNA and proteins, and combinations thereof. In an embodiment, the at least one coded polypeptide sequence is formed by chemical-based peptide synthesis, by in vitro translation of at least one recombinant polynucleotide sequence encoding the at least one coded peptide sequence, or a combination thereof. A benefit of embodiments of forming a coded polypeptide sequence by chemical-based synthesis, by in vitro translation, or combinations thereof can be the cost-effective and large-scale production of a wide variety of data storage polymers.

In an embodiment, the present method includes identifying at least one target peptide sequence in the at least one coded polypeptide, wherein the at least one target peptide sequence includes at least one detectable label on a polypeptide N-terminus, at least one detectable label on a polypeptide C-terminus, at least one nucleotide recognition sequence, at least one protease recognition sequence, or a combination thereof and determining the target peptide sequence by mass spectroscopy. Such embodiments make use of multilayer coding mechanisms of biological systems, for example, amino acid coding and nucleotide-peptide interactions, to enable efficient random-access data retrieval through the use of distinct structural motif formations. Such embodiments can not only provide Exabyte data storage capabilities with dramatically lower energy and space requirements but can also provide a benefit of built-in direct random access capability. Such embodiments can also provide benefits of enhanced data security and future data storage sustainability.

In certain embodiments, the at least one nucleotide recognition sequence includes a TALE identification sequence, a zinc finger sequence, a CRISPR recognition sequence, or a combination thereof. In such embodiments, the method includes providing at least one labeled nucleotide recognizing the at least one nucleotide recognition sequence; and identifying a target peptide sequence in the coded peptide sequence by hybridizing the at least one labeled nucleotide to the at least one nucleotide recognition sequence. Application of multi-layer coding by including combinations of peptide-based coding, TALE identification sequences, and nucleic acid recognition sequences, can provide benefits of greater efficiency and accuracy of data storage and retrieval. Such embodiments can provide an additional benefit of greater data security; for example, in order to read the coded peptide sequence into binary code, one will need to determine the coded peptide sequence by mass spectrometry. Additionally, in embodiments wherein at least one target peptide sequence is identified in the at least one coded polypeptide, determining the target peptide sequence requires that one has available not only the recording key, but also the specific nucleotide recognition sequence key. Such embodiments can provide a benefit of data security that is analogous to that of a two-factor authentication scheme. In some applications of such embodiments, one person could possess the recording key, while another person possesses the nucleotide recognition sequence key, so that the two people must communicate together to be able to read the target peptide sequence.

Examples of structural motif formations that can be included in embodiments and systems herein can also include protein-protein interactions such as antibody-antigen epitope binding and receptor-ligand interactions, as well as the binding of transcription factors to specific DNA structures. Such structures can be built into an amino acid coding system to act as guidance structures for random data access capabilities.

In certain embodiments, the method includes storing the at least one coded polypeptide as a lyophilized powder, in a liquid buffer, immobilized on a microarray, or a combination thereof. In various embodiments, a desired coded polypeptide sequence can be determined by an appropriate mass spectrometry method, such as MALDI-TOF mass spectrometry, to determine a coded polypeptide sequence.

In an embodiment, the method provides including at least one nucleotide-binding sequence in the at least one coded polypeptide; immobilizing the at least one coded polypeptide on at least one position in a microarray; providing at least one detectably labeled polynucleotide recognized by the at least one nucleotide-binding sequence; and identifying at least one target coded polypeptide by hybridizing the at least one detectably labeled polynucleotide to the at least one nucleotide-binding sequence. In certain embodiments, the at least one nucleotide-binding sequence includes a TALE identification sequence, a zinc finger sequence, a CRISPR recognition sequence, or a combination thereof. In certain embodiments, the at least one detectably labeled polynucleotide includes a molecular label. Such a molecular label can include a fluorescent label, a luminescent label, a radioactive label, or combinations thereof. In certain embodiments, immobilizing the at least one coded polynucleotide on at least one position in a microarray includes a streptavidin-biotin bond, a polyhistidine tag bound to a silicon, glass, or a metal chip surface, or a combination thereof.

In some embodiments, peptide-based data can be fractioned and loaded on microarray chips, and each fraction can be identified with a TALE identification sequence. The initial sequence of each data fraction on microarray chips can be loaded with a specific TALE. To enable random data access, capture DNA sequences relevant to each TALE sequence can be synthesized and labeled with a fluorescent dye. The data retrieval can be done by mass spectrometry, including, but not limited, to MALDI-TOF mass spectrometry. After hybridization reaction of TALE-DNA, the desired fraction can be illuminated by a fluorescence label. Sequencing of amino acids can begin from the area of fluorescent illumination.

Embodiments herein including immobilizing at least one coded polypeptide on at least one position on a microarray can provide a benefit of the fractionation of large amounts of data, allowing efficient reading of desired sections of the data in parallel, rather than in serial fashion. Such embodiments can provide a benefit of random access to the fractionated data that can be analogous to computer random access memory. Such embodiments can also provide a benefit of substantially reducing the cost of peptide sequencing for data retrieval.

Embodiments herein including at least one TALE identification sequence can provide the benefits of the flexible design and coding capacity of TALE motifs, providing the capacity for the design of a potentially unlimited number of TAL-DNA tags, and providing a main advantage of the TALE-oligonucleotide recognition system compared to antigen-antibody recognition. TALE identification motifs can be used as identification tags in various types of microarray systems as well. TALE identification motifs can provide a benefit of the capacity of quick access to information of the desired data fraction, without the need for sequencing of whole coding sequences.

Embodiments herein disclose a polypeptide storage system including at least one coded polypeptide made by embodiments of the methods herein.

An embodied method of recording and reading a binary code herein includes providing a binary code; creating a recording key by assigning at least two amino acids or at least two nucleic acid residues a binary code identity; recording the binary code into at least one coded polypeptide or at least one polynucleotide by adding the at least two amino acids or the at least two nucleic acid residues in sequence to form a coded peptide sequence or a coded nucleotide sequence according to the recording key, wherein the coded peptide sequence or the coded nucleotide sequence corresponds to the binary code; determining the coded peptide sequence or the coded nucleotide sequence by mass spectroscopy; and reading the coded peptide sequence or the coded nucleotide sequence into the binary code by identifying the at least two amino acids according to their binary code identity.

Embodiments herein can include a series of recording and reading a binary code in one or more layers of encoding. For example, a binary code can be encoded in an amino acid sequence and read into a binary code in more than one repeated round of encoding and reading, using one or more recording keys in sequence. Such embodiments can provide a benefit of enhanced data security by including multiple layers of data encryption requiring multiple keys. In some embodiments, multiple layers or types of nucleotide binding sequences can be incorporated into the recording and reading of the binary code, providing a benefit of enhanced data security by requiring knowledge of how many and which types of nucleotide binding sequences were used to encode the data. Embodiments of the methods disclosed herein can include a series or reading and recording steps that include post-recording modification of the polymer sequence in a manner analogous to that of post-translational modification of polypeptides. For example, a polypeptide sequence could undergo glycosylation or phosphorylation. One benefit of this sort of post-recording modification could be enhanced data security because it would be necessary to know how the polymer sequence was modified before it could be read.

Embodiments herein can include systems for recording and reading a binary code that incorporates one or more tamper proof or tamper resistant elements that can provide a response or an alarm if an attempt to hack the data is made. Such an element can include an enzyme that can destroy all the information encoded in polypeptide or oligonucleotide sequences. Suitable enzymes for destroying the information encoded in a polypeptide and oligonucleotides include trypsin and endonucleases for DNA and RNA, respectively. Such an element can include an opening key that is specific for a container in which the encoded polypeptide sequences are stored.

### Embodiments of Systems and Methods for Solving a Polynomial Time Problem

Where there are numerous different possible conditions for formation of networks, biological solutions may also be used for solving NP hard problems, such as biologically inspired algorithms, genetic algorithms, and DNA computing algorithms, especially when the number of nodes increases. See, e.g., Adleman L M, Molecular computation of solutions to combinatorial problems, Science, 266:1021-4 (1994), and Qian et al., Scaling up digital circuit computation with DNA strand displacement cascades, Science, 332:1196-201 (2011), the entire contents of which are hereby incorporated by reference herein.

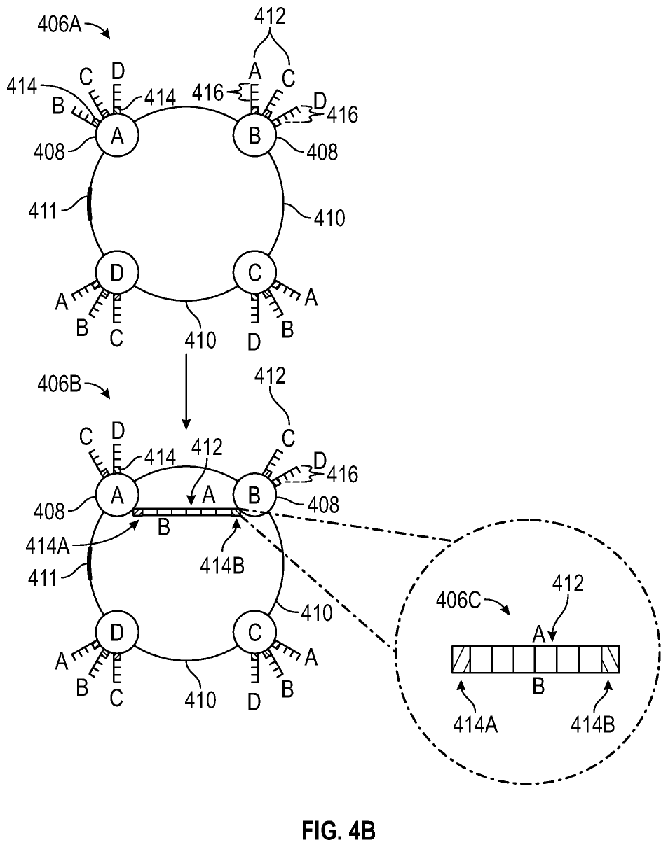

The present disclosure relates to systems for solving a polynomial time problem. FIG. 4A schematically represents an example traveling salesman problem (TSP) 400. The non-limiting example presented in FIG. 4A has 4 nodes (A, B, C, and D) 402 connected by 6 routes (roads) 404 of varying distances between cities, each of which may be traveled in either direction between cities. The traveling salesman problem starts at a given city 402, and travels along routes 404 of various distances (5, 6, 3, 11, 7, and 13) such that each other city is visited exactly once before the route ends back at the starting city 402. The paths start and end with city A. Cities are defined by A-D circles.

FIG. 4B is a schematic depiction of an embodiment of a closed molecular loop structure for solving the polynomial time problem depicted in FIG. 4A. In this non-limiting example, the closed molecular loop structure 406A includes 4 (N) nodes corresponding to cities A, B, C, and D having N number of map locations, including each of the N nodes connected to a different node by an oligomer containing chain 410, which physically connects all nodes in a network together (regardless of the distances between the nodes). There is a junction area 411 within the oligomer containing chain 410, which is capable of being recognized by restriction enzymes. The junction area 411 allows for nodes to be added to or subtracted from the system or the closed molecular loop structure.

Each node is connected to N−1 different single stranded oligonucleotide sequences (412) which have a length or number of based pairs that is representative of a distance between node (cities). In FIG. 4B, a single stranded oligonucleotide sequences 412 is designated A, B, C, or D, depending on which node they have a complementary strand such that each single stranded oligonucleotide identification sequence contains an identification portion 414 corresponding to an identity of the node to which it is attached (example 414A attached to Node A; 414B attached to Node B), and an interaction portion 416 complementary to one single stranded oligonucleotide identification sequence on another node. For example, Node A would bind to Node B then the structures 406B would be formed, when the interaction portion 412 B on Node A hybridized with the interaction portion 412 A on Node B, wherein the interaction portions would form a double stranded portion between the interaction portions of A and B, a magnified view of which is shown in 406C. The hybridized single stranded oligonucleotide sequences or double stranded oligonucleotide identification sequence has a length that is proportionate to the distance between map locations A and B. At any point, the double stranded segment can be recognized by a protein as TALE, zinc fingers, and a recognition unit in Crisper, allowing for the attachment of detectable probes, such as a fluorescent molecule for qualitative or quantitative analysis.

The present disclosure relates to systems and methods for solving a polynomial time problem using a molecular based system. In an embodiment, a system for solving a polynomial time problem a polynomial time problem and a map, wherein the map includes N number of map locations with a distance between their map locations; and a closed loop molecular structure having a number N of nodes located along the closed loop molecular structure, wherein each of the N nodes corresponds to a different map location, wherein each of the N nodes is connected to a different node by an oligomer containing chain, wherein each of the nodes is connected to N−1 different single stranded oligonucleotide identification sequences, wherein each single stranded oligonucleotide identification sequence contains an identification portion, wherein the identification portion contains a sequence which corresponds to an identity of the node to which it is attached, and an interaction portion, which is complementary to one single stranded oligonucleotide identification sequence on another node, wherein each pair of single stranded oligonucleotide identification sequences that is capable of hybridizing with its complementary single stranded oligonucleotide identification sequence, to form a double stranded oligonucleotide identification sequence between a pair of nodes, has a length corresponding to the distance between the map location of the pair of nodes.

In various embodiments of a system for solving a polynomial time problem, the single stranded oligonucleotide identification sequences include single stranded DNA, a RNA, a single stranded polymer, or combinations thereof. In certain embodiments, the oligomer or oligomer containing chain includes amino acids, nucleic acids, polyethylene glycol, an acrylate polymer, a water-soluble polymer, or combinations thereof. In certain embodiments, the closed loop molecular structure is dissolved in an aqueous buffer solution containing at least one polar buffer, a hydrogel, or a combination thereof. In certain embodiments, the nodes include polymer microbeads, carbon nanotubes, carbon nanoparticles, polypeptides, and combinations thereof. In certain embodiments, the nodes are connected to the single stranded oligonucleotide identification sequences including a streptavidin-avidin bond, an overlapping polynucleotide handle, or a combination thereof. In certain embodiments, at least one oligomer containing chain includes at least one restriction enzyme recognition site, at least one protease cleavage site, or combination thereof. In certain embodiments, at least one oligomer containing chain contains a junction area, wherein the junction area is a sequence of oligonucleotide capable of being recognized by a restriction enzyme. One benefit of such a junction area can be that the junction area facilitates the addition or subtraction of node to the closed loop molecular structure.

Embodiments herein provide methods of solving a polynomial time problem. In an embodiment, a method includes providing a polynomial time problem, a map, and a closed loop molecular structure of a system for solving a polynomial time problem disclosed herein, wherein the molecular structure is in an aqueous buffer solution; forming double stranded oligonucleotide identification sequences between the nodes, wherein the double stranded oligonucleotide identification sequences have a length that correlates to the map distance between the nodes; heating the aqueous buffer solution at a heating rate to a measurement temperature; and adding a double stranded detection molecule to the aqueous buffer at a measurement time. In an embodiment, the method can further include sequencing the double stranded oligonucleotide identification sequences present at the measurement time by mass spectroscopy to provide the sequences of the identification portions of a pair of nodes; correlating the sequence of the identification portion to the pair of nodes they identified; quantifying an amount of the identification portions for each pair of nodes; and generating an answer to the polynomial time problem by correlating the amount of the identification portions for each pair of nodes present at the measurement time with the answer to the polynomial time problem. In an embodiment, the method can include detecting the double stranded detection molecule to quantify an amount or relative amount of the double stranded oligonucleotide identification sequences present at the measurement time. Suitable detection method can include fluorescent spectroscopy, UV-vis spectroscopy, and Geiger counter. In an embodiment, the method can include continuously monitoring formation of the double stranded detection molecule at various times during heating or cooling.

In some embodiments, the method includes providing at least two sample vessels containing the molecular structure in an aqueous buffer solution; forming double stranded oligonucleotide identification sequences between the nodes in the at least two sample vessels at room temperature, wherein the double stranded oligonucleotide identification sequences include at least one nucleotide-binding sequence selected from a TALE identification sequence, a zinc finger sequence, a CRISPR recognition sequence, or a combination thereof, wherein the double stranded detection molecule is selected from a TALE DNA recognition domain, a zinc finger, a CRISPR-cas9 recognition domain, or a combination thereof; and sequencing the double stranded oligonucleotide identification sequences present in the at least two sample vessels at the least two measurement times by mass spectroscopy to provide the sequences of the identification portions of pairs of nodes.

In an embodiment, the method includes labeling the double stranded detection molecule before adding the double stranded molecule to the aqueous buffer at the measurement time; and detecting a signal from the labeled double stranded detection molecule before sequencing the double stranded oligonucleotide identification sequences present at the measurement time.

In an embodiment, the at least two sample vessels are connected to one or more microfluidic systems. In such embodiments, the one or more microfluidic systems can be controlled by one or more computer programs to help manage the recording and reading steps. Similarly, in such embodiments, the one or more microfluidic systems can be controlled by one or more computer programs to manipulate the various components necessary for solving a polynomial time problem as disclosed herein.

In certain embodiments, the double stranded oligonucleotide identification sequences between the nodes, wherein the double stranded oligonucleotide identification sequences have a length that correlates to the map distance between the nodes. In an embodiment, the correlation between the length of the double stranded oligonucleotide identification sequence and the distance between the pair of nodes connected is a proportion or a ratio of length to distance.

In an embodiment, the method includes making a molecular computer through an automated programmable micro-fluidic system. The microfluidic system in such an embodiment can provide the hardware for the molecular computer. In some embodiments, the molecular coding units are dissolved in liquid buffers and are stored in separate containers. In some embodiments, at least one container is connected to a micro-fluidic system. In some embodiments, the micro-fluidic system injects the molecular coding units into a new container to create the answer pool for the polynomial time problem.

### EXAMPLES

### Example 1 Method of Recording and Reading a Binary Code

### Example 1A. Conversion of Binary Coding to Peptide-Based Coding System

The first step to accomplish the successful storage of data using an amino acid-based system is to convert the binary 0 and 1 format to amino acid sequences using the conversion method shown in Table 1:

TABLE 1
Conversion of binary data to amino acid format

| | | |
|---|---|---|
| Binary Conversion | Amino Acid | Code |

| | | | | | | |
|---|---|---|---|---|---|---|
| 1 | 0 | 0 | 0 | 0 | Asp (D) | D |
| 2 | 0 | 0 | 0 | 1 | Ser (S) | S |
| 3 | 0 | 0 | 1 | 1 | Leu (L) | L |
| 4 | 0 | 1 | 1 | 1 | Pro (P) | P |
| 5 | 1 | 1 | 1 | 1 | His (H) | H |
| 6 | 1 | 0 | 0 | 1 | Gln (Q) | Q |
| 7 | 1 | 0 | 0 | 0 | Arg [R] | R |
| 8 | 1 | 1 | 0 | 0 | Glu [E] | E |
| 9 | 1 | 1 | 1 | 0 | Thr (T) | T |
| 10 | 1 | 0 | 1 | 1 | Ala (A) | A |
| 11 | 1 | 1 | 0 | 1 | Val (V) | V |
| 12 | 0 | 1 | 0 | 0 | Lys (K) | K |
| 13 | 0 | 0 | 1 | 0 | Asn (N) | N |
| 14 | 0 | 1 | 1 | 0 | Phe (F) | F |
| 15 | 0 | 1 | 0 | 1 | Cys {C} | C |
| 16 | 1 | 0 | 1 | 0 | Trp (W) | W |

All digital data is already available in a binary 0 and 1 format. Referring to FIG. 3 as an example, we show the result of converting of text, from a binary 0 and 1 format to an amino acid format using the conversion system presented in Table 1. To demonstrate the compaction capacity of the amino acid format, we include a snapshot of the same data in binary (0 and 1) form, referring to FIG. 3.

Once the amino acid sequence representing the original text has been transcribed, the sequence will be synthesized via standard peptide synthesis techniques. For Phase I, we limit the peptide length to about 100 amino acid sequences. This will allow for the cost-effective synthesis of peptides with high accuracy. For Phase II we will optimize the peptide length based on the technical and economical parameters learned in phase I.

After synthesis, the resulting amino acid sequence will be stored in lyophilized form at ambient temperature for 3 months. Once the data is needed the sample is sent for peptide sequencing and converted back to 0 and 1 binary code according to Table 1. Sequencing will be conducted on a monthly schedule during the 3 months. We will determine the success of this portion by achieving a less than 95% error rate between the starting and converted binary code.

We also will construct a multi-layer coding system to allow random direct access to data through the use of specific protein recognition motifs, such as Zinc finger and TALE coding sequences.

### Example 1B. Designing a Multi-Layer Coding System for Random Data Access

To address the need for fast access to the information, in addition to the coding sequences, we designed amino acid sequences that can be recognized and tracked through specific protein—DNA interactions. Peptides (with the length of 100 amino acids) were synthesized applying a chemical peptide synthesis method.

Biological systems apply multiple layers of coding for data storage and processing. several examples of different coding layers in biological systems include DNA (made of four coding subunits), peptide/proteins (made of amino acids' coding subunits), Zinc finger-DNA binding coding systems, TALE-DNA binding coding systems, systemic hormones, and neurotransmitters in the neural system. There are three main classes of DNA-protein binding systems including zinc fingers, TALEs, and mRNA guide CRISPR-CAS systems. DNA binding proteins have been applied for several research and clinical purposes including site-specific genetic targeting. Zinc finger domain consists of approximately 30 amino acids in a ββα configuration, with the DNA-binding residues of each zinc finger localized within a short contiguous stretch of residues, designated positions −1, 3, and 6, on the surface of the zinc finger α-helix. The side-chains of these residues interact with the major groove of DNA to make specific contacts, typically with three nucleotides.

Transcription activator-like Effectors (TALEs) are natural bacterial effector proteins used by Xanthomonas sp. to modulate gene transcription in host plants to facilitate bacterial colonization. The central region of the protein contains tandem repeats of amino acids sequences (termed monomers) that are required for DNA recognition and binding. Although the sequence of each monomer is highly conserved, they differ primarily in two positions termed the repeat variable diresidues (RVDs). Recent reports have found that the identity of these two residues determines the nucleotide binding specificity of each TALE repeat and a simple cipher specifies the target base of each RVD (NI=A, HD=C, NG=T, NN=G or A). Thus, each monomer targets one nucleotide and the linear sequence of monomers in a TALE specifies the target DNA sequence in the 5′ to 3′ orientation. The natural TALE binding sites within plant genomes always begin with a thymine, which is presumably specified by a cryptic signal within the non-repetitive N-terminus of TALEs. The tandem repeat DNA binding domain always ends with a half-length repeat. Therefore, the length of DNA sequence being targeted is equal to the number of full repeat monomers plus two.

TALEs provide a special advantage, in that each coding unit of TALE can recognize one nucleotide. This unique property provides a very flexible and specific recognition capacity. Since each cipher specifically targets one nucleotide, it provides a coding system between TALEs and the matching DNA. This allows us to be able to design a specific TALE for almost any DNA sequence. This high flexibility provides a great advantage for TALE-DNA recognition in experimental assays that require a large number of screenings (such as quick access to different data partitions in protein/peptide-based data storage systems). A specific TALE sequence will be identified for each data partition. Initial data of each partition will be tagged with a specific TALE. Each data partition can be retrieved quickly by addition of the matching DNA sequence of each TALE that has been labeled with a fluorescent dye. The data retrieval will be done by mass spectrometry. After a hybridization reaction of TALE-DNA, the desired fraction will be illuminated by fluorescence labeling. Sequencing of amino acids will start from the area of fluorescent illumination, referring to FIG. 2. On-chip sequencing service will be provided by CHROMATRAP®, US.

### Example 1C. Constructing Customized TALE Sequences

Due to the nature of the repetitive nature of TALEs, firstly we generate libraries of DNA-binding monomers. Then we apply a hierarchical ligation strategy to assemble monomeric units together. Plasmid libraries of TALE monomeric units will be synthesized by applying the ADDGENE® TALE Toolbox kit (Cat #1000000019). Assembling of monomeric units will be done by different combinations of monomeric application of a Golden Gate method included in the ADDGENE® TALE Toolbox kit.

Briefly, we first amplify each nucleotide-specific monomer sequence with ligation adaptors that uniquely specify the monomer position within the TALE tandem repeats. Once this monomer library is produced, it can conveniently be re-used for the assembly of many TALEs. For each TALE desired, the appropriate monomers are first ligated into hexamers, which are then amplified via the polymerase chain reaction (PCR). Then, a second Golden Gate digestion-ligation with the appropriate TALE cloning backbone yields a fully assembled, sequence-specific TALE. The backbone contains a ccdB negative selection cassette flanked by the TALE N and C-termini, which is replaced by the tandem repeat DNA-binding domain when the TALE has been successfully constructed. ccdB selects against cells transformed with an empty backbone, therefore yielding clones with tandem repeats inserted.

### Example 1D. Constructing TALE monomer libraries

TALE monomer plasmid pNI_v2, pNG_v2, pNN_v2 and pHD-V2 will be purchased from ADDGENE®. TALE Toolbox PCR Primers for TALE construction will be purchased from Integrated DNA Technologies®. Hercules II Fusion polymerase will be applied for the polymerase chain reaction (Agilent Technologies®, Cat #600679). Plasmids of TALE monomers will be amplified by polymerase chain reactions to make a library. Assembling of different combinations of TALE monomers can be applied to generate unique identification units before the coding units. Subsequently, to verify the monomer amplification is done successfully, gel electrophoresis will be done. To this end, 2% agarose gel in 1×TBE electrophoresis buffer with 1× Syber safe dye will be prepared.

### Example 1E. Assembling Custom Designed TALE Identification Sequences

In order to design specific target sequences firstly, we need to consider that typical TALE recognition sequences are identified in the 5′ to 3′ direction and begin with a 5′ thymine. The procedure below describes the construction of TALEs that bind a 20 bp target sequence (5′T0N1N2N3N4N5N6N7N8N9N10N11N12N13N14N15N16N17N18N19-3′, where N=A, G, T, or C), where the first base (typically a thymine) and the last base are specified by sequences within the TALE backbone vector. The middle 18 bp are specified by the RVDs within the middle tandem repeat of 18 monomers according to the cipher NI=A, HD=C, NG=T, and NN=G or A.

In the first stage, N1-N18 sequences will be divided into sub-sequences of length 6 (N1N2N3N4N5N6, N7N8N9N10N11N12, and N13N14N15N16N17N18). For example, a TALE targeting 5′-TGAAGCACTTACTTTAGAAA-3′ can be divided into hexamers as (T) GAAGCA CTTACT TTAGAA (A), where the initial thymine and final adenine (in parenthesis) are encoded by the appropriate backbone. In this example, the three hexamers will be: hexamer 1=NN-NI-NINN-HD-NI, hexamer 2=HD-NG-NG-NI-HD-NG, hexamer 3=NG-NG-NINN-NI-NI. Due to the adenine in the final position, we will use one of the NI backbones: pTALE-TF_v2(NI) or pTALEN_v2(NI). Subsequently, assembling hexamers will be done using Golden Gate digestion-ligation. Briefly, to perform a simultaneous digestion-ligation (Golden Gate) reaction to assemble each hexamer the following reagents will be added to each hexamer tube (Table 2):

TABLE 2
Reagents for assembling of TALE
hexamers applying Golden Gate kit

| Compound | Amount | Final Concentration | |
|---|---|---|---|
| Esp3 (Bsm B1) | 0.76 | 0.375 | U/μl |
| Tango Buffer 10X | 1 | 1X | |
| Dithiothreitol | 1 | 1 | mM |
| (DTT) 10 mM | | | |
| T7 Ligase 3000 | 0.251 | 75 | U/μl |
| U/μl | | | |
| ATP 10 mM | 1 | 1 | mM |
| 6 monomers | 6 × 1 | | |
| Total | 10 | | |

### Example 1F. Expression and Isolation of TALE Identification Sequences

After assembly of TALE monomeric units, each TALE gene construct will be transferred to a protein expression vector. Also, to facilitate the protein isolation process, the expression vector containing a Biotin tag will be used. To this end, the PinPoint™Xa Protein Purification System (Promega®, Cat #V2020) will be applied. Protein expression will be done according to the manufacturers' instructions. TALE domains will be isolated by applying the Biotin Affinity Purification kit (THERMO FISHER SCIENTIFIC®, C21386). Each TALE tag will be loaded at the beginning of a specific data partition on the microarray chips.

A specific oligonucleotide sequence which is matching with each TALE sequence (also called a capture oligonucleotide) will be synthesized separately (ILLUMINA®, US) and will be conjugated to a fluorescent dye for the next step analysis. In the next step, each labeled oligonucleotide will be applied for detection of its relevant data fraction.

### Example 2. Method of Solving a Polynomial Problem

Referring to FIG. 5A, the TSP problem may have multiple nodes 502 which define multiple routes 504 resulting in a number of possible outcomes. Referring to FIG. 5B, the system 506 can be constructed to process the possible outcomes and determine an optimal solution to the TSP problem in FIG. 5A. For example, nodes A-D representing cities A, B, C, and D at their respective map locations can be constructed from polymer microbeads. A closed loop molecular structure can be constructed connecting each of the nodes to a different node by an oligomer containing chain composed of an amino acid water-soluble polymer. Each oligomer containing chain is constructed to have a length between connected nodes that corresponds to the distance between corresponding city map locations. The polymers can be attached to the nodes via streptavidin-avidin bonds. Single stranded DNA identification sequences can be attached to each node such that each node is connected to N−1 different single stranded DNA identification sequences. Each single stranded DNA identification sequence is constructed to include an identification portion containing a sequence corresponding to the identity of the node to which it is attached, and an interaction portion complementary to one single stranded DNA identification sequence on another node.

The system can be used in a method for solving the TSP problem in FIG. 5A, to provide a solution to the problem that may be defined to meet certain criteria, such as the shortest and/or least expensive route to travel to four different cities on a map. The closed loop system can be dissolved in an aqueous buffer solution in each of a series of sample tubes and allowed to form double stranded DNA identification sequences between the nodes in the samples at room temperature. The buffer solutions are heated at a heating rate to reach a measurement temperature. When the measurement temperature is reached, a labeled double stranded DNA detection TALE recognition domain molecule is added to the aqueous buffer in each sample at each of a series of measurement times. The TALE detection molecule will specifically attach to the recognized double stranded DNA sequences; signals from these bound molecules will be detected in order to determine an optimal solution to the TSP problem. The double stranded oligonucleotide identification sequences present at the measurement time will be sequenced by mass spectroscopy or automated DNA sequencing to provide the sequence of the identification portions of a pair of nodes. The sequences of the identification portions will be correlated to the pairs of nodes they identified. The amount of the identification portions for each pair of nodes is quantified, and an answer to the TSP problem is generated by correlating the amount of the identification portions for each pair of nodes present at the measurement time with the answer to the TSP problem.

What is claimed is:

1. A method of recording and reading a binary code comprising:
providing a binary code;
creating a recording key by assigning at least two amino acids a binary code identity;
recording the binary code into at least one coded polypeptide by adding the at least two amino acids in sequence to form a coded peptide sequence according to the recording key, wherein the coded peptide sequence corresponds to the binary code;
determining the coded peptide sequence by mass spectroscopy; and
reading the coded peptide sequence into the binary code by identifying the at least two amino acids according to their binary code identity.

2. The method of claim 1, wherein from two to sixteen amino acids are assigned a binary code identity; or
the at least one coded polypeptide sequence is formed by chemical-based peptide synthesis, or by in vitro translation of at least one recombinant polynucleotide sequence encoding the at least one coded peptide sequence, or a combination thereof.

3. The method of claim 1, further comprising:
identifying at least one target peptide sequence in the at least one coded polypeptide, wherein the at least one target peptide sequence includes at least one detectable label on a polypeptide N-terminus, at least one detectable label on a polypeptide C-terminus, at least one nucleotide recognition sequence, at least one protease recognition sequence, or a combination thereof; and
determining the target peptide sequence by mass spectroscopy.

4. The method of claim 3, wherein the at least one nucleotide recognition sequence includes a TALE identification sequence, a zinc finger sequence, a CRISPR recognition sequence, or a combination thereof.

5. The method of claim 3, further comprising:
providing at least one labeled nucleotide recognizing the at least one nucleotide recognition sequence; and
identifying a target peptide sequence in the coded peptide sequence by hybridizing the at least one labeled nucleotide to the at least one nucleotide recognition sequence.

6. The method of claim 1, further comprising storing the at least one coded polypeptide as a lyophilized powder, in a liquid buffer, immobilized on a microarray, or a combination thereof.

7. The method of claim 1, further comprising:
including at least one nucleotide-binding sequence in the at least one coded polypeptide;
immobilizing the at least one coded polypeptide on at least one position in a microarray;
providing at least one detectably labeled polynucleotide recognized by the at least one nucleotide-binding sequence; and
identifying at least one target coded polypeptide by hybridizing the at least one detectably labeled polynucleotide to the at least one nucleotide-binding sequence.

8. The method of claim 7, wherein the at least one nucleotide-binding sequence includes a TALE identification sequence, a zinc finger sequence, a CRISPR recognition sequence, or a combination thereof wherein the at least one detectably labeled polynucleotide includes a molecular label; or wherein immobilizing the at least one coded polynucleotide on at least one position in a microarray includes a streptavidin-biotin bond, a polyhistidine tag bound to a silicon, glass, or a metal chip surface, or a combination thereof.

9. A polypeptide storage system comprising: at least one coded polypeptide made by the process of claim 1.

10. A method of recording and reading a binary code comprising:
providing a binary code;
creating a recording key by assigning at least two amino acids or at least two nucleic acid residues a binary code identity;
recording the binary code into at least one coded polypeptide or at least one polynucleotide by adding the at least two amino acids or the at least two nucleic acid residues in sequence to form a coded peptide sequence or a coded nucleotide sequence according to the recording key, wherein the coded peptide sequence or the coded nucleotide sequence corresponds to the binary code;
determining the coded peptide sequence or the coded nucleotide sequence by mass spectroscopy; and
reading the coded peptide sequence or the coded nucleotide sequence into the binary code by identifying the at least two amino acids according to their binary code identity.

11. A system for solving a polynomial time problem comprising:
a polynomial time problem and a map, wherein the map includes N number of map locations with a distance between their map locations; and
a closed loop molecular structure having a number N of nodes located along the closed loop molecular structure,
wherein each of the N nodes corresponds to a different map location,
wherein each of the N nodes is connected to a different node by an oligomer containing chain,
wherein each of the nodes is connected to N−1 different single stranded oligonucleotide identification sequences,
wherein each single stranded oligonucleotide identification sequence contains an identification portion, wherein the identification portion contains a sequence which corresponds to an identity of the node to which it is attached, and
an interaction portion, which is complementary to one single stranded oligonucleotide identification sequence on another node,
wherein each pair of single stranded oligonucleotide identification sequences that is capable of hybridizing with its complementary single stranded oligonucleotide identification sequence, to form a double stranded oligonucleotide identification sequence between a pair of nodes, has a length corresponding to the distance between the map location of the pair of nodes.

12. The system of claim 11, wherein the single stranded oligonucleotide identification sequences include single stranded DNA, a RNA, a single stranded polymer, or combinations thereof.

13. The system of claim 11, wherein the oligomer includes amino acids, nucleic acids, polyethylene glycol, an acrylate polymer, a water-soluble polymer, or combinations thereof.

14. The system of claim 11, wherein the closed loop molecular structure is dissolved in an aqueous buffer solution containing at least one polar buffer, a hydrogel, or a combination thereof.

15. The system of claim 11, wherein the nodes include polymer microbeads, carbon nanotubes, carbon nanoparticles, polypeptides, and combinations thereof.

16. The system of claim 11, wherein the nodes are connected to the single stranded oligonucleotide identification sequences including a streptavidin-avidin bond, an overlapping polynucleotide handle, or a combination thereof.

17. The system of claim 13, wherein at least one oligomer containing chain includes at least one restriction enzyme recognition site, at least one protease cleavage site, or combination thereof.

18. A method of solving a polynomial time problem comprising:
providing the polynomial time problem, the map, and the closed loop molecular structure of claim 12, wherein the molecular structure is in an aqueous buffer solution;
forming double stranded oligonucleotide identification sequences between the nodes;
heating the aqueous buffer solution at a heating rate to a measurement temperature;
adding a double stranded detection molecule to the aqueous buffer at a measurement time;
sequencing the double stranded oligonucleotide identification sequences present at the measurement time by mass spectroscopy to provide the sequences of the identification portions of a pair of nodes;
correlating the sequence of the identification portion to the pair of nodes they identified;
quantifying a value of the identification portions for each pair of nodes; and
generating an answer to the polynomial time problem by correlating the amount of the identification portions for each pair of nodes present at the measurement time with the answer to the polynomial time problem.

19. The method of claim 18, further comprising:
providing at least two sample vessels containing the molecular structure in an aqueous buffer solution;
forming double stranded oligonucleotide identification sequences between the nodes in the at least two sample vessels at room temperature, wherein the double stranded oligonucleotide identification sequences include at least one nucleotide-binding sequence selected from a TALE identification sequence, a zinc finger sequence, a CRISPR recognition sequence, or a combination thereof, wherein the double stranded detection molecule is selected from a TALE DNA recognition domain, a zinc finger, a CRISPR-cas9 recognition domain, or a combination thereof; and
sequencing the double stranded oligonucleotide identification sequences present in the at least two sample vessels at the least two measurement times by mass spectroscopy to provide the sequences of the identification portions of pairs of nodes.

20. The method of claim 18, further comprising:
labeling the double stranded detection molecule before adding the double stranded molecule to the aqueous buffer at the measurement time; and
detecting a signal from the labeled double stranded detection molecule before sequencing the double stranded oligonucleotide identification sequences present at the measurement time.
